(19) United States
(12) Patent Application Publication
(10) Pub. No.: US 2013/0245463 A1
(43) Pub. Date: Sep. 19, 2013

(54) SYSTEM AND METHOD FOR DISPLAYING PHYSIOLOGICAL INFORMATION

(71) Applicants: Susan Stuebe, Waukesha, WI (US); Wilbur Cruz Colaco, Mumbai (IN); Judy Moore, Hertfordshire (GB); Nicola Telling, Hertfordshire (GB); Juan Antonio Vidal Wagner, Madrid (ES)

(72) Inventors: Susan Stuebe, Waukesha, WI (US); Wilbur Cruz Colaco, Mumbai (IN); Judy Moore, Hertfordshire (GB); Nicola Telling, Hertfordshire (GB); Juan Antonio Vidal Wagner, Madrid (ES)

(21) Appl. No.: 13/610,073

(22) Filed: Sep. 11, 2012

(30) Foreign Application Priority Data
Sep. 12, 2011 (IN) 3117/CHE/2011

(51) Int. Cl. A61B 5/00; A61B 5/01; A61B 5/04; A61B 5/0205

(52) U.S. Cl. CPC A61B 5/743; A61B 5/7475; A61B 5/0205; A61B 5/4356; A61B 5/01; A61B 5/04; A61B 5/7282
USPC 600/483; 600/300; 600/588; 600/549; 600/485; 600/529; 600/508

(57) ABSTRACT

A method of displaying physiological information in a patient monitoring system is provided. The method includes receiving a user input as a digital annotation, maintaining the digital annotation as at least a part of patient monitoring data and displaying the digital annotation concurrently with information displayed by the patient monitoring system.

### BACKGROUND OF THE INVENTION

Embodiments of the subject matter disclosed herein generally relate to a patient monitoring system and more particularly to a fetal monitoring system displaying fetal monitoring data.

Conventional systems for displaying physiological information may be used to monitor physiological characteristics of an individual in real-time. For example, such systems may be used in cardiotocography, electrocardiography, electroencephalography, electromyography, electronystagmography, and polygraphy (i.e., lie detection). Similar systems may also be used to display seismic activity. The systems typically include a roll of strip paper having a pattern of visual indicators (e.g., gridlines), a writing system that makes traces along the strip of paper, and sensors that are connected to the writing system. The sensors may be, for example, attached to an individual at predetermined locations of the body. As the paper is rolled out at a predetermined speed, the writing system makes traces onto the paper that are indicative of the detected signals obtained through the sensors. The visual indicators, the predetermined speed, and the traces may be in accordance with established standards so that a user may quickly review and analyze the information.

However, in some cases, it may be necessary for the system to produce and for the user to review a significant amount of paper. For example, it may be necessary for a clinician to review approximately one meter of paper of a cardiotocograph to analyze conditions during pregnancy. This large amount of paper may be costly and difficult to manage.

Further, it is cumbersome for the user to annotate the chart paper displaying the cardiotocograph. Users are used to using a pen and making notes on the paper strip. Also, searching the large paper displaying the cardiotocograph to find the annotations that the users are interested in is manual and time consuming.

Thus, it may be desirable to provide the information through a digital viewer or display without the production of paper. There is also a need to provide easy annotation methods to record, analyze and search information displayed in such digital viewers.

BRIEF DESCRIPTION OF THE INVENTION

The above-mentioned shortcomings, disadvantages and problems are addressed herein which will be understood by reading and understanding the following specification.

According to an embodiment of the present invention, a method of displaying information in a patient monitoring system is provided. The method comprises receiving a user input as a digital annotation, maintaining the digital annotation as at least a part of patient monitoring data, and displaying the digital annotation concurrently with information displayed by the patient monitoring system.

According to another embodiment of the present invention, a method of searching information in a patient monitoring system is provided. The method comprises launching a user configuration dialog, displaying a list of annotation types available for search, the list of annotation types comprising at least one annotation, receiving a user input, the user input depicting the selected annotation for search, searching the patient monitoring data for the selected annotation and displaying one or more selected annotations upon finding the selected annotation in the patient monitoring data.

According to another embodiment of the present invention, a system for displaying physiological information is provided. The system comprises a user interface having a viewable chart portion configured to display physiological information of an individual and a waveform module configured to obtain physiological signals as a function of time, the waveform module configured to plot a waveform that is based upon the physiological signals on the virtual graph. Further, the user interface is configured to receive digital annotations input by a user, display the waveform and the digital annotation in the chart portion and save the waveform and the digital annotation as a part of patient monitoring data.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a block diagram of a system for displaying physiological information as described in an embodiment;

FIG. 2 shows a flow diagram depicting a method of displaying information in a patient monitoring system as described in an embodiment; and

FIG. 3 shows a flow diagram depicting a method of searching information in a patient monitoring system as described in an embodiment.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

Embodiments described herein may generate a virtual strip chart (VSC) that is similar to a paper strip chart used in various industries. For example, embodiments described herein may generate a virtual cardiotocograph, a virtual electrocardiograph, a virtual electroencephalograph, a virtual polygraph, a virtual electromyograph, a virtual electronystagmograph, or a virtual seismograph. The VSCs may be saved or stored in a database. As used herein, users of the systems, methods, and user interfaces described herein include doctors or clinicians, nurses, patients, researchers, or other systems. The user may review the waveform information as it is generated or the user may review a history of the waveform information.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments that “comprise,” “have,” or “include” an element or a plurality of elements that have a particular property may also include additional such elements that do not have that particular property. Furthermore, when an element is described as being based on a factor or parameter, the term “based on” should not be interpreted as the factor or parameter being the sole factor or parameter, but may include the possibility that the element is also based on other factors or parameters.

As used herein, each of the terms “waveform signals” and “physiological signals” may include only one type of signals or multiple types of signals. For examples, physiological signals may include physiological signals relating to a first type (e.g., fetal heart rate signals) and physiological signals relating to a second type (e.g., intrauterine pressure signals). When multiple types of waveform signals are illustrated as different waveforms, the different waveforms may be synchronized in a predetermined manner. For example, the different physiological signals may be plotted along the same time axis so that the user can correlate an event or condition associated with the first type of physiological signals to an event or condition associated with the second type of physiological signals.

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. For example, one or more of the functional blocks (e.g., modules, processors, or memories) may be implemented in a single piece of hardware (e.g., a general purpose signal processor or random access memory, hard disk, or the like). Similarly, programs may be stand alone programs, may be incorporated as subroutines in an operating system, may be functions in an installed software package, may be a software surface package that is run from a computer server remotely, and the like. It should be understood that the various embodiments are not limited to the arrangements and instrumentality shown in the drawings.

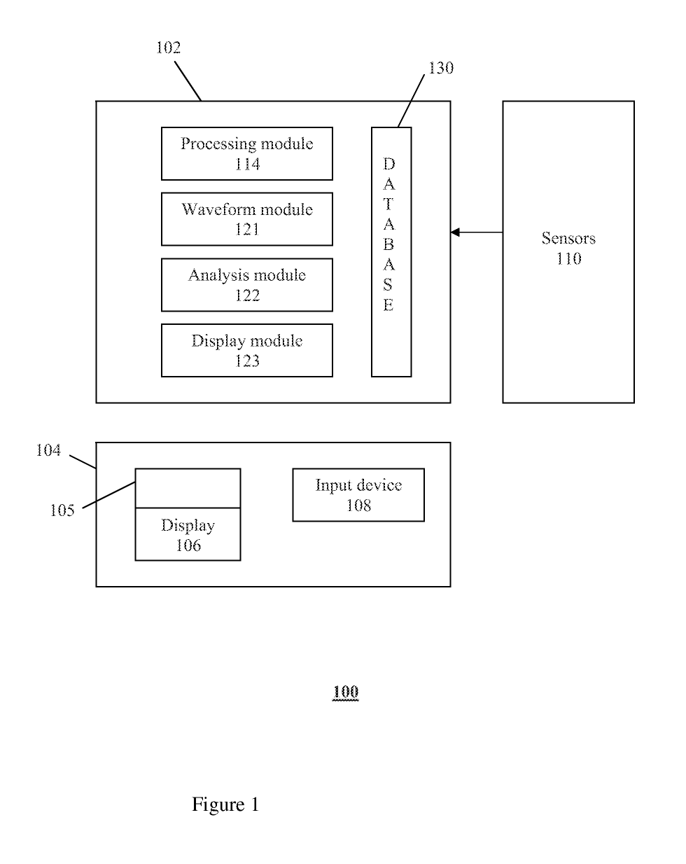

In one embodiment, a system for displaying physiological information is provided. The system comprises a user interface having a viewable chart portion configured to display physiological information of an individual and a waveform module configured to obtain physiological signals as a function of time, the waveform module configured to plot a waveform that is based upon the physiological signals on the virtual graph. Further, the user interface is configured to receive digital annotations input by a user, display the waveform and the digital annotation in the chart portion and saving the waveform and the digital annotation as a part of patient monitoring data. This is further explained in conjunction with FIG. 1.

FIG. 1 is a block diagram of an exemplary system 100 for displaying waveform information or, more specifically, physiological information. The system 100 includes a computing device or system 102 that is communicatively coupled to a user interface 104. The user interface 104 may include instruments (e.g., user display), hardware, and software (or a combination thereof) that permit the system 100 to display information to the user and, in some embodiments, permit the user to provide user inputs or selections. For example, the user interface 104 may include a display 106 (e.g., monitor, screen, touchscreen, and the like) and an input device 108 (e.g., keyboard, computer mouse, touchscreen, and the like). In some embodiments, a device constituting the input device 108 may also be the device constituting the display 106 (e.g., touchscreen). The display 106 may be configured to show a viewable area that includes a viewable chart portion 105, which is described in greater detail below. The user interface 104 may also be configured to query the user and accept or receive user inputs from a user of the system 100.

The system 100 may be integrated into one component (e.g., a laptop computer) or may be several components that may or may not be located near each other. In alternative embodiments, the computing system 102 may be communicatively coupled to sensors 110 that are configured to detect measurements, such as from an individual (e.g., a patient), and communicate the measurements to the system 100 as waveform signals. In particular embodiments, the measurements are physiological measurements. The sensors 110 may be configured to detect different physiological measurements, such as a heart rate, body temperature, blood pressure, respiratory rate, electrical activity, or intrauterine pressure.

The computing system 102 may include or be part of a server system, a workstation, a desktop computer, a laptop computer, or a personal device, such as a tablet computer or a smart phone. However, the above are only examples and the computing system 102 may be other types of systems or devices. In the illustrated embodiment, the computing system 102 includes a processing module 114, which may comprise a controller, processor, or other logic-based device. The processing module 114 may have or be communicatively coupled to modules for performing methods as described herein. The modules may include a waveform module 121, an analysis module 122 and a display module 123. In addition to the above, there may be several other modules or sub-modules of the processing module 114 that are not shown. Each of the modules 121-123 may be communicatively coupled to a memory or database 130 via, for example, the Internet or other communication network. Although the database 130 is shown as being shared by the modules 121-123, each module 121-123 may have a separate memory or database.

The waveform module 121 may be configured to obtain the waveform signals. For example, the measurements detected by the sensors 110 may be transmitted to the waveform module 121. Optionally, the waveform module 121 may convert or modify the waveform signals so that the signals are recognized by other modules in the system 100 for further manipulation or analysis. For example, the waveform module 121 may identify the waveform signals as intrauterine pressure signals or fetal heart rate signals and convert or modify the physiological signals so that the signals are recognized by the other modules as corresponding to intrauterine pressure or a fetal heart rate. In other embodiments, the system 100 may be configured such that the measurements obtained through the sensors 110 are assumed to relate to certain measurements. Furthermore, in other embodiments, the user may instruct the system 100 that the signals obtained through certain sensors 110 relate to certain measurements. Also, in some cases, the waveform module 121 may receive the waveform signals from a database or another system or device. For example, the measurements may not be directly detected from a patient in real-time. Instead, the measurements may be stored and transmitted to the waveform module 121 for follow-up analysis or research.

The display module 123 may operate in conjunction with the waveform module 121. In some embodiments, the display module 123 may include the waveform 121. The display module 123 may store various parameters that may be used for displaying the waveform signals. In some embodiments, the display module 123 may store various parameters that are used by established standards for displaying the physiological information. For example, the United States standard for cardiotocography has a signal range along the vertical axis of between 30 and 240 beats/minute (bpm). The U.S. scaling is 30 bpm/cm, and the recording speeds may be 1, 2, and 3 cm/min. On the other hand, the international standard for cardiotocography has a signal range along the vertical axis of between 50 and 210 bpm. The international scaling is 20 bpm/cm, and the recording speeds may also be 1, 2, and 3 cm/min.

The analysis module 122 is configured to analyze the waveform signals and identify any events-of-interest. In some embodiments, the analysis module 122 may automatically analyze the waveform signals before the waveform signals are used to generate the plotted waveform. In other embodiments, the analysis module 122 may analyze the plotted waveform generated by the waveform module 121. For example, as the system 100 receives physiological measurements from the sensors 110 that relate to intrauterine pressure and a fetal heart rate, the analysis module 122 may analyze the waveform signals directly or the plotted waveform to determine if predetermined patterns have occurred. The analysis module 122 may use one or more algorithms to identify the events-of-interest. If an event-of-interest is identified, the analysis module 122 may generate an alert or notify the user in some manner.

The database 130 may store data that can be retrieved by the components or modules of the system 100 and other remotely located systems through the Internet or other communication network. The database 130 can store data that the modules 121-123 require in order to accomplish the functions of the modules 121-123. For example, the database 130 can store the waveform signals obtained from the sensors 110.

The modules 121-123 (and the processing module 114) include one or more processors, microprocessors, controllers, microcontrollers, or other logic-based devices that operate based on instructions stored on a tangible and non-transitory computer readable storage medium. For example, the modules 121-123 may be embodied in one or more processors that operate based on hardwired instructions or software applications. The database 130 can be or include electrically erasable programmable read only memory (EEPROM), simple read only memory (ROM), programmable read only memory (PROM), erasable programmable read only memory (EPROM), FLASH memory, a hard drive, or other type of computer memory.

The physiological signals may relate to an individual (e.g., patient) that may be an animal or human. For example, the physiological signals may relate to an intrauterine pressure or fetal heart rate. In some embodiments, the physiological signals are obtained directly from a patient in real-time through sensors. In other embodiments, the physiological signals may be obtained through a local or remote database 130.

The waveform module 121 may acquire the physiological signals through sensors 110 or through local or remote database 130 and subsequently plot a waveform using the variables in the physiological signals. The waveform is displayed in a visual strip chart (VSC). The waveform has a time dimension or axis and a signal dimension or axis that extend in directions that are perpendicular to each other. The waveform may have a pattern of visual cues or indicators that facilitate a user's analysis of the waveform.

A user may enter user inputs through the user interface 104 to request a history of the VSC. The user may enter the requested time period (e.g., from the beginning of recordings, the last ten minutes) to recall the history of the VSC or, in embodiments where the user interface 104 includes a touchscreen, the user may slide a finger in a direction that is along the time dimension to scroll through the VSC. When a history of the VSC is recalled, the waveform module 121 may provide the plotted waveform. In some embodiments, when a user is reviewing the history of the VSC, the system 100 may continue to obtain the physiological signals.

Embodiments described herein may include VSC's that are configured to include user-generated annotations or system-generated notices that are overlaid onto the waveform. The annotations and notices may facilitate analyzing and/or displaying the physiological information. Furthermore, the annotations and notices may be stored and recalled with the waveforms. The annotations may be generated based on user inputs. For example, a user may add an annotation to the VSC to facilitate determining an average movement or trend of the waveform over an extended period of time. The user may then review a history of the waveform to determine how the physiological signals have changed over an extended period of time.

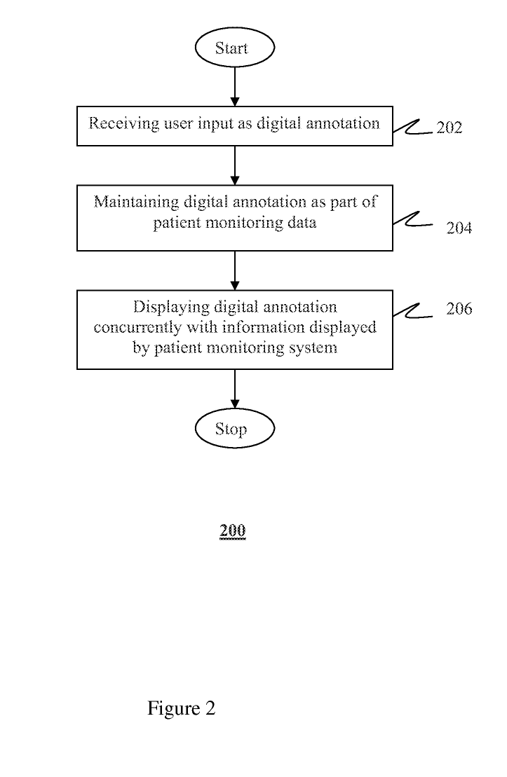

Accordingly, in one embodiment as shown in FIG. 2, a method 200 of displaying information in a patient monitoring system is provided. The method 200 comprises steps of receiving a user input as a digital annotation at step 202, maintaining the digital annotation as at least a part of patient monitoring data at step 204 and displaying the digital annotation concurrently with information displayed by the patient monitoring system at step 206.

The annotation may comprise a shaded (or colored) band or zone. The annotation may be provided by, for example, a clinician to indicate where the waveform should be located. For instance, upon reviewing the VSC, the clinician may determine that a “safe” range for the fetal heart rate extends approximately between 90-120 bpm. If the waveform (i.e., fetal heart rate) moves outside of this range, the system may generate an alarm to notify the clinician or other user. Accordingly, the annotation may be defined by alarm limits. If the waveform exceeds the upper alarm limit (120 bpm) or is less than the lower alarm limit (90 bpm), the user of the system may be notified. For example, the system may generate an audible noise that is heard in the room of the patient or in another remote room (e.g., nurse's station) or the system may page or somehow electronically notify the user.

A digital annotation may include a written note, an image that is being displayed by the patient monitoring system, a predefined annotation or a written note and/or information copied from another source.

The annotation may be located in the blank area. The annotation may include text provided by a user of the system. The text may be used to inform the user, such as one who subsequently reviews the VSC that an event occurred at the noted time. For example, the annotation may be text that states “changed patient position” or “provided medication.” The text may provide information that explains why the waveform changed or if the patient responded at all to an event. This feature allows the user to make annotations on findings at the time the event is identified. Further, the annotation thus entered can be stored with the patient monitoring data.

The system 100 may determine (e.g., through the analysis module 122) that the waveform exceeded the upper alarm limit of the user-selected annotation. The system 100 analyzes the waveforms using the analysis module 122 to determine whether the event is concerning. The annotations may be stored with the waveforms. As such, when reviewing the history of the VSC, a user could review the waveforms in addition to the annotations made by a user or any notices provided by the system.

A summary report may be generated periodically or when requested by the user. The summary report may be displayed or automatically printed or sent electronically to a user of the system (e.g., via text or email). The summary report may include rows and columns with cells having information that summarize a health of a patient at the time that the summary report was generated. In one embodiment, annotation input by the user may be included in the summary report generated by the system 100.

In one exemplary embodiment, a method of making annotations on a visual strip chart of an electronic fetal monitoring machine is described. The visual strip chart is capable of receiving user inputs and when activated launches various methods to annotate a fetal monitoring data. The user is given the opportunity to annotate the data dynamically as the data is being read. Upon activating the VSC, the user can annotate the fetal monitoring data using a virtual keyboard or by using predefined annotations, or by entering a reporting tab where the user can file findings to the report.

Using a virtual keyboard tab, the user types on the virtual keyboard to input his/her findings. Using the predefined annotations tab, the user chooses from a set of canned sentence fragments to create an interpretation of his findings. The user can also create his/her own predefined annotations that can be saved in the system for future use.

Using the report input tab, the user can identify information regarding the contractions, risks, baseline rate, accelerations, decelerations, variability, and an assessment and action plan. Upon entering this information into the form, the data is embedded into the summary report that is displayed to the user at the end of the session.

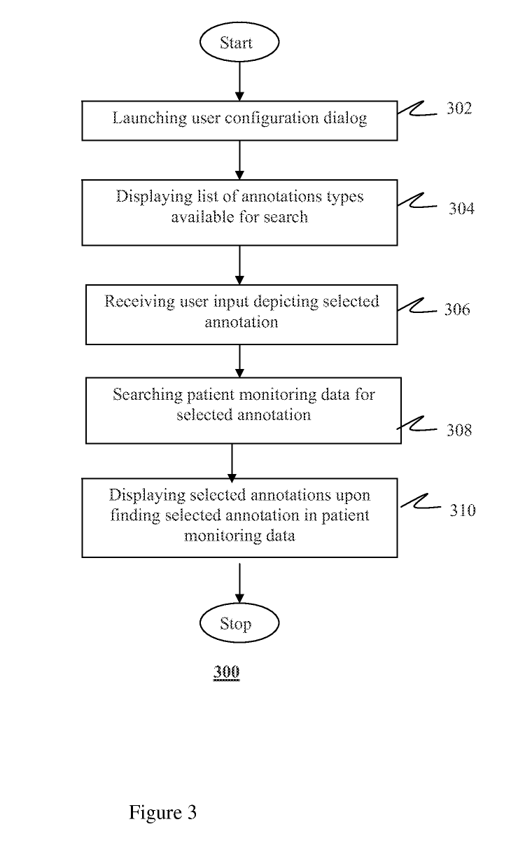

In another embodiment as shown in FIG. 3, a method 300 of searching information in a patient monitoring system is provided. The method 300 comprises steps of launching a user configuration dialog at step 302, displaying a list of annotations types available for search at step 304, the list of annotation types comprising at least one annotation, receiving a user input at step 306, the user input depicting the selected annotation for search, searching the patient monitoring data for the selected annotation at step 308 and displaying one or more selected annotations upon finding the selected annotation in the patient monitoring data at step 310.

Optionally, the method 300 may include receiving a user input to retrieve a history of the plotted waveform. For example, the user may communicate to the system 100 that the user would like to review that past 20 minutes of recorded waveform signals. Upon receiving the user inputs, the plotted waveform may be retrieved. The plotted waveform may begin moving from the selected time. In some embodiments, the user may use a touchscreen that allows the user to scroll back and forth through the history. For example, the system may allow the user to review a history of the physiological signals at a rate that is faster than the recording speed.

Also optionally, the method 300 may include receiving user inputs to provide a user-generated annotation onto the waveform. The annotations may be stored by the system so that the annotations may be retrieved along with the plotted waveform. The method 300 may also include notifying the user that an event-of-interest has been identified by the system 100. For example, the system 100 may identify a predetermined pattern that is associated with an event-of-interest. The system 100 may then notify the user that a possible event-of-interest has occurred. The method 300 may also include generating a summary report of the physiological signals including the user-generated annotations as described above.

In one exemplary embodiment, the method 300 allows the user to view specific annotations marked on a fetal monitoring strip. When the user designates certain search criteria, the system finds that type of annotation and allows the user to view one or more selected annotations. The user can move from one selected annotation to another by pressing a control key.

When the user launches the User Configuration dialog, the annotation tab supplies a list of events the user can search on. The user selects the annotations that the user wishes to search on. When the user makes the selection, the VSC displaying fetal monitoring data moves from one selected annotation to another selected annotation that are found on the fetal monitoring data. In order to return to the default setting of the controls or end/change the search criteria, the user returns to the configuration dialog and unselects the annotation buttons.

This utility allows the users to enter more robust data into the fetal monitoring data that is being displayed on the VSC. The ease of making decision on the type of annotation the user wishes to search on, and the ability to find this type of annotation is an advantage.

At least one technical effect of various embodiments includes displaying physiological information along with annotations input by a user in a virtual manner that may be reviewed and analyzed by a user. Another technical effect includes displaying physiological information that is similar to established display standards of strip paper charts so that the user may quickly review and analyze the physiological information. Another technical effect includes storing a history of the physiological signals along with recorded annotations so that a user may retrieve or recall the physiological signals along with the annotations to review at least a portion of the time that the physiological signals were obtained.

Embodiments of the invention allow users to annotate using contemporary computer interactions that are easy to input, review, analyze and archive. The ease of use significantly expedites the workflow and provides a more efficient and comprehensive workflow that allows for more accurate monitoring. The methods 200 and 300 described herein allow the user to extrapolate more data from the VSC easily.

This written description uses examples to describe the subject matter herein, including the best mode, and also to enable any person skilled in the art to make and use the subject matter. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of displaying information in a patient monitoring system, the method comprising:
receiving a user input as a digital annotation;
maintaining the digital annotation as at least a part of patient monitoring data; and
displaying the digital annotation concurrently with information displayed by the patient monitoring system.

2. The method of claim 1, wherein the digital annotation includes a written note.

3. The method of claim 1, wherein the digital annotation includes an image being displayed by the patient monitoring system.

4. The method of claim 1, wherein the digital annotation includes a predefined annotation.

5. The method of claim 1, wherein the digital annotation includes both a written note and information copied from another source.

6. The method of claim 1, further comprising saving a current image of patient monitoring data being displayed by the patient monitoring system, and saving the digital annotation as being associated with the patient monitoring data.

7. The method of claim 1, further comprising receiving information from an additional user input as an additional digital annotation.

8. A method of searching information in a patient monitoring system, the method comprising:
launching a user configuration dialog;
displaying a list of annotation types available for search, the list of annotation types comprising at least one annotation;
receiving a user input, the user input depicting a selected annotation for search;
searching patient monitoring data for the selected annotation; and
displaying one or more selected annotations upon finding the selected annotation in the patient monitoring data.

9. A system for displaying physiological information, the system comprising:
a user interface having a viewable chart portion configured to display physiological information of an individual; and
a waveform module configured to obtain physiological signals as a function of time, the waveform module configured to plot a waveform that is based upon the physiological signals,
wherein the user interface is configured to receive digital annotations input by a user, display the waveform and the digital annotation in the chart portion and save the waveform and the digital annotation as a part of patient monitoring data.

10. The system of claim 9, wherein the waveform module is configured to store a history of the plotted waveform, the user interface being configured to receive user inputs for retrieving the history of the plotted waveform and displaying the history of the plotted waveform in the chart portion.

11. The system of claim 9, wherein the waveform module is configured to store a history of the plotted waveform along with the digital annotation input by the user, the user interface being configured to receive user inputs for retrieving the history of the plotted waveform along with the digital annotation and displaying the history of the plotted waveform along with the digital annotation in the chart portion.

12. The system of claim 9, further comprising sensors communicatively coupled to the waveform module, the sensors configured to detect the physiological signals from the individual.

13. The system of claim 9, further comprising an analysis module, the analysis module configured to identify predetermined patterns of the waveform that are indicative of an event-of-interest, the analysis module configured to notify the user about the occurrence of the event-of-interest.

14. The system of claim 9, wherein the digital annotation input by the user and the plotted waveform have a fixed relationship with one another.

15. The system of claim 9, wherein the physiological signals include a fetal heart rate and intrauterine pressure.

16. The system of claim 9, wherein the physiological signals relate to a heart rate, body temperature, blood pressure, respiratory rate, electrical activity, or intrauterine pressure.
